(19) United States
(12) Patent Application Publication
(10) Pub. No.: US 2023/0190523 A1
(43) Pub. Date: Jun. 22, 2023

(54) SMART WIRELESSLY DRIVEN CONTACT LENS FOR MEASURING INTRAOCULAR PRESSURE OF AND TREATING GLAUCOMA PATIENTS

(71) Applicants: PHI BIOMED INC., Seoul (KR); POSTECH ACADEMY-INDUSTRY FOUNDATION, Gyeongsangbuk-do (KR)

(72) Inventors: Sei Kwang Hahn, Seoul (KR); Sang Baie Shin, Gyeongsangbuk-do (KR); Tae Yeon Kim, Gyeongsangbuk-do (KR)

(21) Appl. No.: 17/610,682

(22) PCT Filed: Sep. 4, 2020
(86) PCT No.: PCT/KR2020/011909

§ 371 Date: Nov. 11, 2021

(51) Int. Cl. A61F 9/00; A61B 3/16; A61B 5/00; A61K 9/00; A61K 31/5377; A61K 31/5575; G02C 7/04

(52) U.S. Cl. CPC A61F 9/0017; A61B 3/16; A61B 5/4839; A61K 9/0009; A61K 9/0051; A61K 31/5377; A61K 31/5575; G02C 7/04; A61B 2562/0261; A61B 2562/0285; G02C 11/10

(57) ABSTRACT

The present invention provides a wirelessly driven contact lens including a strain sensor capable of detecting an increase in intraocular pressure in real time and a drug reservoir capable of lowering the intraocular pressure by releasing a drug based on the increase in intraocular pressure. In the present invention, there may be provided a personal therapy system that measures intraocular pressure in real time and properly releases a therapeutic drug according to the intraocular pressure that is measured through the strain sensor and the drug reservoir for releasing a drug based on an intraocular pressure state of a glaucoma patient.

### TECHNICAL FIELD

The present invention relates to a wirelessly driven contact lens for monitoring intraocular pressure and treating glaucoma in a glaucoma patient based on an intraocular pressure state.

### BACKGROUND

Glaucoma is one type of eye disease, in which optic nerve cells are gradually damaged due to a continuous increase in intraocular pressure, and can be fatal. Glaucoma may permanently affect vision, may slowly and continuously progress, and ultimately worsen. Therefore, there is a need for a system capable of regularly and accurately diagnosing and treating glaucoma in real time.

Intraocular pressure is one of the most important factors in diagnosing glaucoma and is the most important risk factor related to glaucoma. Therefore, there is a need for a system that can precisely diagnose an increase in intraocular pressure through a continuous and non-invasive method, and there is a need for a new method of promptly treating an increase in intraocular pressure.

In the existing glaucoma treatment system that has been developed, since a drug is not administered based on an intraocular pressure state, there is a serious problem related to side effects of drugs. However, since changes in intraocular pressure and the states of intraocular pressure based on glaucoma are different depending on the individual, a drug should be administered in an appropriate amount and at an appropriate cycle for each individual. In addition, since the existing intraocular pressure measurement system that has been developed constitutes a single system that enables only diagnosis, there is a limitation in that a cycle of treatment cannot be interrupted.

Therefore, there is a need for a personal therapy system that measures an intraocular pressure state of a glaucoma patient and controls the administration of a drug based on the intraocular pressure state that is measured.

### Technical Problem

Since degrees of intraocular pressure change of glaucoma patients are different depending on the individual, there is a need for a personal therapy system that treats glaucoma through accurate diagnosis based on an intraocular pressure state of a patient.

However, glaucoma therapy systems that has been developed so far have side effects of drug treatment because it is difficult to administer a drug based on an intraocular pressure state of a patient.

Accordingly, the present invention is directed to providing a wirelessly driven contact lens for monitoring intraocular pressure and treating glaucoma in a glaucoma patient, to which a high sensitivity and high transparency strain sensor for measuring intraocular pressure is coupled in order to minimize side effects of a drug and maximize a treatment effect. The present invention is also directed to providing a personal therapy system using the wirelessly driven contact lens.

### Technical Solution

According to an embodiment of the present invention, a wirelessly driven contact lens for monitoring intraocular pressure and treating glaucoma in a glaucoma patient includes a strain sensor which is transparent and measures intraocular pressure of a subject, and a drug reservoir.

The strain sensor and the drug reservoir may be formed on a transparent substrate.

The strain sensor may measure a change in resistance due to a change in intraocular pressure, and when an abnormality is detected in the change in intraocular pressure, a drug may be released from the drug reservoir.

According to another embodiment of the present invention, a method of manufacturing the above-described wirelessly driven contact lens includes forming a sacrificial layer soluble in water on a handling substrate,

forming a transparent substrate on the sacrificial layer,

forming a strain sensor and a drug reservoir on the transparent substrate, and

transferring the transparent substrate, on which the strain sensor and the drug reservoir are formed, into a contact lens.

According to still another embodiment of the present invention, a system for monitoring intraocular pressure and treating glaucoma in a glaucoma patient includes a wirelessly driven contact lens including a strain sensor which is transparent and measures intraocular pressure of a subject, and a drug reservoir, and

smart glasses configured to wirelessly transmit or receive an electrical signal and control driving of the strain sensor and the drug reservoir of the wirelessly driven contact lens.

The strain sensor and the drug reservoir may be formed on a transparent substrate.

The strain sensor may measure a change in resistance using electrical connection and disconnection between nanomaterials according to a change in curvature of an eyeball due to a change in intraocular pressure, and when an abnormality is detected in the change in intraocular pressure, a drug may be released from the drug reservoir.

According to yet another embodiment of the present invention, a method of treating glaucoma based on an intraocular pressure state using the above-described system includes applying, by a strain sensor in a wirelessly driven contact lens, a constant voltage to an eyeball of a subject for a predetermined measurement time and wirelessly measuring a change in current according to a change in resistance, and

when the change in current due to a change in intraocular pressure of the eyeball of the subject is measured to be greater than or equal to a set range, dissolving gold of an electrode pattern sealing a drug well of a drug reservoir through chlorine ions to form AuCl⁴⁻ and opening the drug reservoir.

### Advantageous Effects

According to the present invention, a wirelessly driven contact lens for monitoring intraocular pressure and treating glaucoma in a glaucoma patient includes a strain sensor capable of detecting an increase in intraocular pressure in real time and a drug delivery system capable of lowering the intraocular pressure by releasing a drug according to the increase in intraocular pressure.

In the present invention, through the strain sensor and the drug delivery system, intraocular pressure can be measured in real time, and a therapeutic drug can be appropriately released from a contact lens. Accordingly, personal therapy is possible through a feedback treatment system based on an intraocular pressure state of a glaucoma patient.

In addition, a transparent strain sensor is used so as not to obstruct a patient's field of vision, and a flexible drug reservoir (i.e., a drug delivery system) is used to prevent loss of a therapeutic drug, thereby minimizing side effects of the drug and maximizing a glaucoma treatment effect.

Furthermore, communication can be performed non-invasively and conveniently through smart glasses capable of wirelessly communicating with the outside.

### DESCRIPTION OF DRAWINGS

FIG. 1 shows images showing a principle in which a strain sensor of a wirelessly driven contact lens measures intraocular pressure through a change in curvature of an eyeball according to a change in intraocular pressure.

FIG. 2 shows images showing a structure of a drug delivery system in a wirelessly driven contact lens.

FIG. 3 shows electrical characteristics in a process in which a gold thin film of a drug delivery system dissolves through an electrochemical reaction with chlorine ions in a living body.

FIG. 4 is an image of a drug release channel formed by patterning a transparent substrate through reactive ion etching.

FIG. 5 is an image showing a drug release channel opening as a gold thin film of a drug delivery system dissolves through an electrochemical reaction with chlorine ions.

FIG. 6 shows images showing wireless energy supply and wireless communication of a wirelessly driven contact lens for measuring intraocular pressure of a patient and releasing an appropriate drug based on an intraocular pressure state.

FIG. 7 shows images showing processes of forming a drug delivery system according to the present invention.

FIG. 8 shows photographs showing flexibility of a drug delivery system according to the present invention.

FIGS. 9 and 10 are images showing a wirelessly driven contact lens to which a strain sensor formed according to the present invention is coupled.

FIG. 11 shows photographs showing a wirelessly driven contact lens to which a strain sensor capable of being wirelessly driven is coupled.

FIG. 12 shows results of measuring intraocular pressure using a strain sensor according to the present invention.

FIG. 13 shows results of measuring a change in intraocular pressure of a rabbit through a wirelessly driven contact lens to which a strain sensor is coupled.

FIG. 14 is a graph showing absorbance of a drug released by applying an electrical signal to a drug delivery system according to the present invention.

### BEST MODES OF THE INVENTION

The present invention relates to a wirelessly driven contact lens for monitoring intraocular pressure and treating glaucoma in a glaucoma patient, the wirelessly driven contact lens including a transparent strain sensor for measuring intraocular pressure of a subject, and a drug reservoir.

The strain sensor and the drug reservoir are formed on a transparent substrate.

The strain sensor measures a change in resistance due to a change in intraocular pressure, and when an abnormality is detected in the change in intraocular pressure, a drug is released from the drug reservoir.

Hereinafter, the wirelessly driven contact lens for monitoring intraocular pressure and treating glaucoma in a glaucoma patient according to the present invention will be described in more detail.

In the present invention, the strain sensor for measuring intraocular pressure of a glaucoma patient is coupled to the wirelessly driven contact lens, thereby measuring intraocular pressure and treating glaucoma in the glaucoma patient according to a change in intraocular pressure.

The wirelessly driven contact lens of the present invention is based on at least one polymer selected from the group consisting of an elastomer such as a silicone elastomer, a silicone hydrogel, and a polymer hydrogel of poly(2-hydroxyethyl methacrylate) (PHEMA), polyvinylpyrrolidone (PVP), poly(lactic acid-glycolic acid) (PLGA), or polyvinyl alcohol (PVA).

In the present invention, the transparent substrate is formed inside the wirelessly driven contact lens, and the strain sensor and the drug reservoir are formed on the transparent substrate.

The transparent substrate has characteristics such as excellent light transmittance, excellent flexibility and elasticity, and excellent biocompatibility. The transparent substrate may include at least one selected from the group consisting of parylene C, polydimethylsiloxane (PDMS), a silicone elastomer, polyethylene terephthalate (PET), and polyimide (PI).

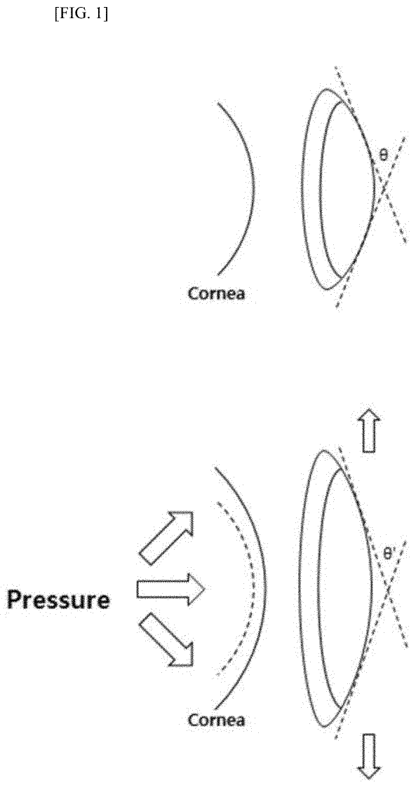

In the present invention, the strain sensor may be a transparent sensor that measures intraocular pressure of a subject and may measure a change in resistance due to a change in intraocular pressure. Specifically, FIG. 1 shows images showing a principle in which the strain sensor of the wirelessly driven contact lens measures intraocular pressure. As shown in FIG. 1, the strain sensor may measure intraocular pressure through a change in resistance of the sensor according to a change in curvature of an eyeball.

In one embodiment, the strain sensor may include a nanomaterial layer formed on the transparent substrate and a passivation layer formed on the nanomaterial layer. In this case, the strain sensor may be formed on a surface of the transparent substrate which faces toward an eyeball.

The nanomaterial layer includes nanomaterials, and the nanomaterials may be materials having biocompatibility. Specifically, the nanomaterials may include at least one selected from the group consisting of zero-dimensional materials that are nanoparticles, one-dimensional nanomaterials that are nanowires, nanofibers, or nanotubes, and two-dimensional nanomaterials that are graphene, MoS₂, or nanoflakes. More specifically, the nanomaterials may include silver (Ag) and/or gold (Au). More specifically, the nanomaterials may be silver-gold core-shell nanowires (Ag@Au core-shell NWs).

In addition, the passivation layer may include a component having excellent elasticity and flexibility and having biocompatibility. More specifically, the passivation layer may include at least one selected from the group consisting of parylene C, PDMS, a silicone elastomer, PET, and PI.

In one embodiment, the structure of the strain sensor is not particularly limited and may include a circle or a straight line. Specifically, the strain sensor may have a structure that entirely or partially surrounds the cornea of an eyeball and may have a circular structure that entirely surrounds the cornea of the eyeball.

In the present invention, since the drug reservoir is sealed by an electrode pattern including gold and operates in conjunction with the above-described strain sensor, when an abnormality is detected in a change in intraocular pressure by the strain sensor, gold in the electrode pattern of the drug reservoir may react with chlorine ions in a living body and dissolve so that a drug may be released from the drug reservoir.

In one embodiment, the drug reservoir may include the electrode pattern including gold and formed on a portion of a surface of the transparent substrate and a drug well layer formed on the electrode pattern and including one or more drug wells with a shape that is recessed so as to face outward. In this case, perforations may be formed in the transparent substrate, and the electrode pattern may cover the perforations.

In one embodiment, a drug may be positioned in the drug well. The drug may be a drug capable of treating glaucoma or may include a drug carrier which may release the drug capable of treating glaucoma, and a drug-release-controlling material.

In the present invention, an antenna may be additionally formed on the transparent substrate in addition to the above-described strain sensor and drug reservoir. The antenna may be formed to be coplanar with the strain sensor on the transparent substrate.

The antenna may transmit and receive power and signals to and from the outside through induced current and electromagnetic resonance.

In one embodiment, the antenna may be a circular antenna having a circular structure.

In one embodiment, the antenna may be made of nanomaterials, and the nanomaterials may include at least one selected from the group consisting of metal thin film materials, zero-dimensional materials that are nanoparticles, one-dimensional nanomaterials that are nanowires, nanofibers, or nanotubes, and two-dimensional nanomaterials that are graphene, MoS₂, or nanoflakes.

Both the strain sensor and the antenna may be made of nanomaterials, but due to a difference in a pattern structure and a difference in a content of nanomaterials, the strain sensor and the antenna may serve as a strain sensor and an antenna, respectively. For example, an antenna pattern may include Ag@Au core-shell NWs. In this case, the antenna pattern may be formed to be thicker than the strain sensor, and a content of nanomaterials and a length of nanowires thereof may be different from those in the strain sensor, thereby preventing resistance according to a change in intraocular pressure, which is measured by the strain sensor, from changing.

Components constituting the strain sensor, the drug reservoir, and the antenna according to the present invention all have excellent biocompatibility and thus may operate stably in tears and may be harmless to a living body.

In addition, the present invention relates to a method of manufacturing the above-described wirelessly driven contact lens for monitoring intraocular pressure and treating glaucoma in a glaucoma patient.

The wirelessly driven contact lens may include: operation S1 of forming a sacrificial layer soluble in water on a handling substrate;

operation S2 of forming a transparent substrate on the sacrificial layer;

operation S3 of forming a strain sensor and a drug reservoir on the transparent substrate; and

operation S4 of transferring the transparent substrate, on which the strain sensor and the drug reservoir are formed, into a contact lens.

Operation S1 is an operation of forming the sacrificial layer on the handling substrate.

The sacrificial layer may serve as an adhesive layer between the handling substrate and the transparent substrate and may assist in transferring the transparent substrate on which the strain sensor and the drug reservoir are formed. As long as the sacrificial layer is soluble in water, the sacrificial layer is not particularly limited and may include at least one selected from the group consisting of PVA, dextran, and the like.

Operation S2 is an operation of forming the transparent substrate on the sacrificial layer. Since the sacrificial layer serves as an adhesive, the transparent substrate may be easily attached to the handling substrate and may be easily delaminated from the handling substrate by dissolving the sacrificial layer in a subsequent process.

In one embodiment, a material having excellent light transmittance may be used for the transparent substrate, and the above-described type may be used therefor.

Operation S3 is an operation of forming the strain sensor and the drug reservoir on the transparent substrate.

In one embodiment, the strain sensor may be manufactured through operation a1 of forming a mask material for patterning on the transparent substrate;

operation a2 of patterning a sensor and a circuit by coating nanomaterials on the transparent substrate, on which the mask material is formed, through a lift-off process; and

operation a3 of forming a passivation layer on the sensor and circuit that are patterned.

Operation a1 is an operation of forming the mask material for patterning on the transparent substrate.

The mask material may serve as a shadow mask, and the mask material may be used to pattern the nanomaterials. As the mask material, a material usable as a photoresist may be used without limitation.

Operation a2 is an operation of patterning the sensor and the circuit by coating the nanomaterials on the transparent substrate, on which the mask material is formed, through a lift-off process.

Through the above operation, a pattern of the nanomaterials may be formed on the transparent substrate. The above-described types may be used as the nanomaterials, and specifically, Ag@Au core-shell NWs may be used.

The nanomaterials prepared in the above operation may serve as an intraocular pressure sensor, that is, a strain sensor. The strain sensor may be made of nanomaterials, and by using the connection and disconnection between the nanomaterials, the strain sensor may measure a change in resistance of the sensor according to a change in curvature of an eyeball and may measure intraocular pressure through a change in current according to the change in resistance.

In addition, the circuit manufactured in the above operation may serve to connect the strain sensor, the antenna, and the drug reservoir.

Operation a3 is an operation of forming the passivation layer on the sensor and circuit that are patterned.

In the present operation, the passivation layer may be formed to prevent loss of the nanomaterials and improve electrical stability. The passivation layer may include the above-described types of components.

In one embodiment, the drug reservoir may be manufactured through operation b1 of forming an electrode pattern including gold on a portion of a surface of the transparent substrate;

operation b2 of forming a drug well layer including one or more drug wells on the electrode pattern; and

operation b3 of forming a drug release channel in the transparent substrate.

Operation b1 is an operation of forming the electrode pattern including gold on a portion of the surface of the transparent substrate.

In the present invention, the electrode pattern may be formed in a form in which a gold electrode pattern is formed on the transparent substrate using photolithography and positive electrodes of the electrode pattern covers the drug reservoir.

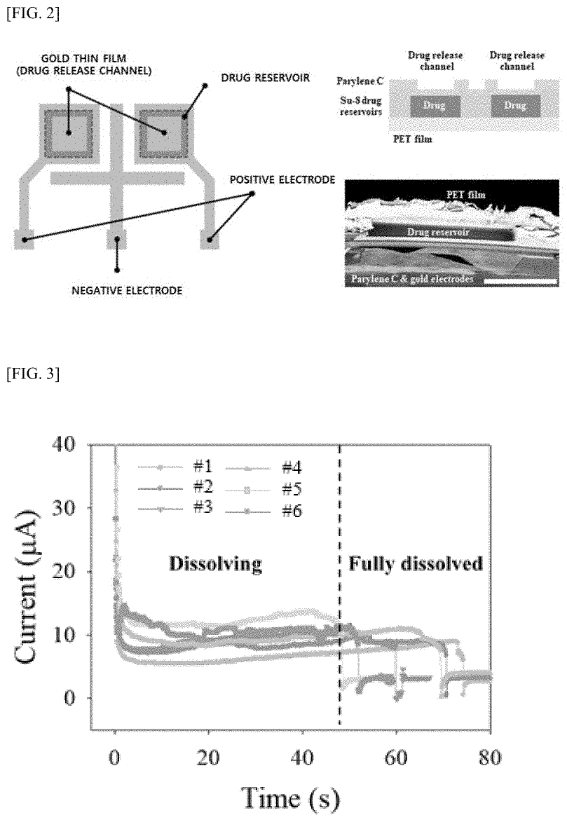

In one embodiment, the electrode pattern stacked on the transparent substrate may include the positive electrodes made of a metal including gold and a negative electrode commonly connected to the positive electrodes (see FIG. 2). The electrodes may have a form in which a plurality of positive electrodes constitute an array. The electrode may be made of gold and titanium in different portions. The gold forming the electrode pattern may be removed by being electrolyzed by a voltage applied in an electrolyte (see FIG. 3). Therefore, the positive electrode of the electrode pattern may be used as a gate of a channel (drug release channel) through which an accommodated drug is delivered by a voltage.

Operation b2 is an operation of forming the drug well layer including one or more drug wells on the electrode pattern. The drug well may store a drug.

The drug well layer including the drug wells may include a flexible and biocompatible component. Specifically, the drug well layer may include at least one selected from the group consisting of PDMS, a silicone elastomer, polyurethane acrylate (PUA), and an SU8. The drug well layer may be formed on the electrode pattern.

Operation b3 is an operation of forming the drug release channel in the transparent substrate.

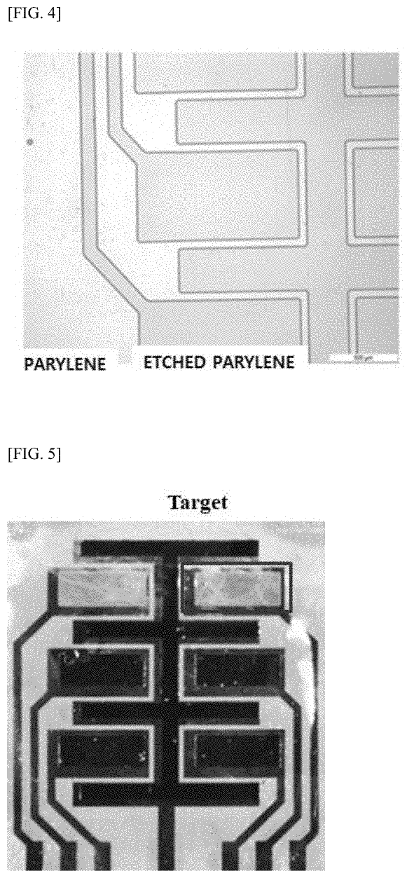

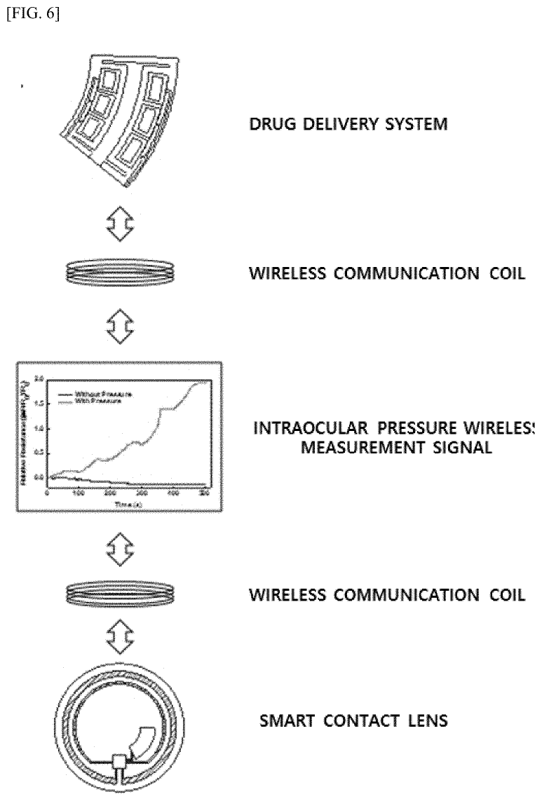

In operation b3, the drug release channel may allow a gold electrode to react with chlorine ions in a living body, and when the gold electrode dissolves through an electrochemical reaction, the drug release channel may serve as a channel though which a drug is released. The drug release channel may be formed by forming a perforation in the transparent substrate, and in this case, the perforation may be formed through reactive ion etching using oxygen (see FIG. 4).

In addition, operation S4 is an operation of transferring the transparent substrate, on which the strain sensor and the drug reservoir are formed, into the contact lens.

The sensor and drug reservoir manufactured on the sacrificial layer may be transferred by dissolving the sacrificial layer in biocompatible water.

In addition, the present invention may further include an operation of forming an antenna on the transparent substrate. The above operation may be performed during operation S3 and may be performed in the same manner as in a method of manufacturing the strain sensor.

In addition, the present invention relates to a wirelessly driven system for monitoring intraocular pressure and treating glaucoma in a glaucoma patient.

The wirelessly driven system for treating glaucoma according to the present invention may include a wirelessly driven contact lens including a transparent strain sensor for measuring intraocular pressure of a subject and a drug reservoir, and smart glasses.

The above-described contact lens may be used as the wirelessly driven contact lens. Specifically, in the wirelessly driven contact lens, the strain sensor and the drug reservoir are formed on a transparent substrate. The strain sensor may measure a change in resistance due to a change in intraocular pressure, and when an abnormality is detected in the change in intraocular pressure, a drug may be released from the drug reservoir.

In one embodiment, the strain sensor and the drug reservoir may be connected to an application-specific integrated circuit (ASIC) chip to enable wireless communication. The strain sensor and the drug reservoir may be driven by receiving an electrical signal transmitted from an external system through the ASIC chip. A result detected by the strain sensor may be transmitted to the external system to store and process data, and the driving of a drug delivery system may be controlled.

In one embodiment, when the drug reservoir receives an electrical signal transmitted from an external system, and gold of an electrode pattern dissolves through chlorine ions in a living body to form AuCl⁴⁻ according to the electrical signal, the electrode pattern may open so that a drug may be released to the outside from the drug reservoir (see FIG. 5).

In the present invention, the smart glasses may wirelessly transmit or receive the electrical signal to control driving of the strain sensor and the drug reservoir of the wirelessly driven contact lens. The present invention may provide the smart glasses which enable micro-unit long distance adjustment using a transparent electrode including nanomaterials, stretchable electronics, a complementary metal-oxide semiconductor (CMOS), a flexible and biocompatible micro electro-mechanical system (MEMS), and nano electro-mechanical system (NEMS) technology.

In the smart glasses, electrical power may be implemented using wireless inductive power transfer (WiTricity) technology, and wireless communication may be performed using Bluetooth, infrared (IR), and radio frequency (RF) communications in the smart glasses.

An operating system (OS) of the smart glasses may use an Android OS, and the smart glasses may be equipped with an OMAP 4430 SoC, a dual-core central processing unit (CPU), and a 4 GB random-access memory (RAM). A display screen may include 640×360 pixels, and a bone conduction transducer may be used for a sound. Functions of an optical sensor, a biosensor, a pressure, a temperature, and an acoustic electromagnetic (EM) sensor may be controlled using a voice through a microphone, and the smart glasses may be paired with a smartphone, a smart watch, or a personal computer (PC). An embedded 100 mAh lithium ion battery may be used for power, and a photocell may be inserted for auto powering. A total weight of the smart glasses may be less than 20 g, and Wi-Fi 802.11b/g, Bluetooth, and micro Universal Serial Bus (USB) may be available. Photos with a resolution of more than 15 MP and videos with a format of more than 720 p may be implemented using a mounted camera.

In one embodiment, a sensor may be driven through an electrical signal transmitted from the smart glasses, and the sensor from which the signal is received may measure a change in resistance using the electrical connection and disconnection between nanomaterials according to a change in curvature of an eyeball due to a change in intraocular pressure. When the sensor detects the change in intraocular pressure, the sensor may transmit a result of the detected change to the smart glasses through RF wireless communication.

In addition, in one embodiment, a drug delivery system may be driven through an electrical signal transmitted from the smart glasses, and when a gold electrode pattern sealing a drug reservoir of the drug delivery system receives the signal, as the gold electrode pattern dissolves through chlorine ions to form AuCl⁴⁻, the drug delivery reservoir may open.

In addition, the present invention relates to a method of treating glaucoma using the above-described wirelessly driven system, and more particularly, to a method of treating glaucoma based on an intraocular pressure state by measuring intraocular pressure of a glaucoma patient.

In the method of treating glaucoma according to the present invention, a strain sensor in a contact lens applies a constant voltage to an eyeball of a subject for a predetermined measurement time to wirelessly measure a change in current according to a change in resistance. When the change in resistance due to a change in intraocular pressure of the eyeball of the subject is measured to be greater than or equal to a set range, the drug reservoir may open as gold of an electrode pattern sealing a drug well of a drug reservoir dissolves through chlorine ions to form AuCl⁴⁻. In this case, the voltage applied to the eyeball may be, for example, in a range of 0.3 volts to 0.7 volts.

In one embodiment, the strain sensor may be driven through an electrical signal transmitted from smart glasses, and the strain sensor from which the signal is received may measure a change in resistance using the electrical connection and disconnection between nanomaterials according to a change in curvature of an eyeball due to a change in intraocular pressure. The strain sensor may transmit a result of the detected change to the smart glasses or the outside through wireless communication.

In one embodiment, the drug reservoir may be driven through an electrical signal transmitted from the smart glasses or the outside, and the smart glasses may analyze the change in current or the change in resistance transmitted through the strain sensor. When an abnormality is detected in a change in intraocular pressure, the smart glasses may transmit an electrical signal to the drug reservoir, and the drug reservoir from which the signal is received may open.

In addition, in one embodiment, power generated from a wireless electric coil of the smart glasses is received by a wireless electric antenna of a wirelessly driven contact lens, and the power that is received can be used to drive a sensor and a drug delivery system under control of an integrated circuit (IC) chip.

### Modes of the Invention

Hereinafter, the present invention will be described in detail through the following Examples. However, the following Examples are for illustrative purposes only, and the present invention is not intended to be limited by the following Examples.

### Examples

### Manufacturing Example 1: Formation of Strain Sensor

A handling substrate was spin-coated with PVA to form a sacrificial layer. Parylene C was deposited to a thickness of 500 nm on the substrate on which the sacrificial layer was formed. Ag@Au core-shell NWs were patterned through spin coating using a shadow mask or lift-off technique, and an end portion of a sensor was coated with high-density Ag@Au core-shell NWs to form an electrode.

### Manufacturing Example 2: Formation of Drug Reservoir

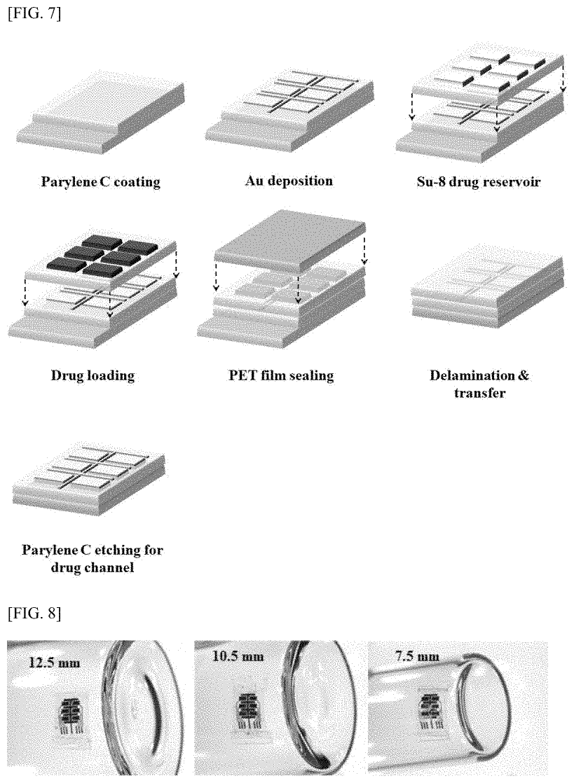

Processes of forming a drug reservoir to be inserted into a contact lens are shown in FIG. 7.

A handling substrate was spin-coated with PVA to form a sacrificial layer, and a parylene layer was coated on the sacrificial layer. Gold was deposited to a thickness of 100 nm on a manufactured transparent substrate to form an electrode

A drug well layer including one or more drug wells was formed on the electrode using an SU8, and PVA (5 wt %) and a drug were mixed in the drug wells, loaded, and then dried.

Thereafter, after a drug reservoir was sealed using a PET film and a parylene layer was deposited to perform passivation, the sacrificial layer was dissolved in water, and then a drug delivery system was transferred.

In order to perforate the transparent substrate of the drug delivery system, a photoresist was formed in a shape of a drug release channel through a photolithography method. After perforations were formed through reactive ion etching using oxygen, the photoresist was removed using acetone.

In the present invention, FIG. 2 shows images showing an electrode structure of a drug reservoir. The electrode structure includes positive electrodes and a negative electrode, and it can be confirmed that a drug well (drug reservoir and drug release channel) is covered with a gold thin film.

In addition, FIG. 8 shows photographs showing measured flexibility of a drug reservoir.

As shown in FIG. 8, it can be confirmed that the drug reservoir has flexibility and thus is applicable to a contact lens.

### Manufacturing Example 3: Manufacture of Contact Lens

A transparent substrate, on which a strain sensor and a drug reservoir were formed, was manufactured. The strain sensor and the drug reservoir were formed on the transparent substrate according to methods of Manufacturing Examples 1 and 2.

The transparent substrate was introduced into a lens manufacturing mold which contained a contact lens manufacturing solution, and constant pressure was applied to perform heat treatment in an oven at about 100° C. for one hour. A contact lens manufactured in the lens manufacturing mold was separated.

The manufactured contact lens includes the strain sensor and the drug reservoir.

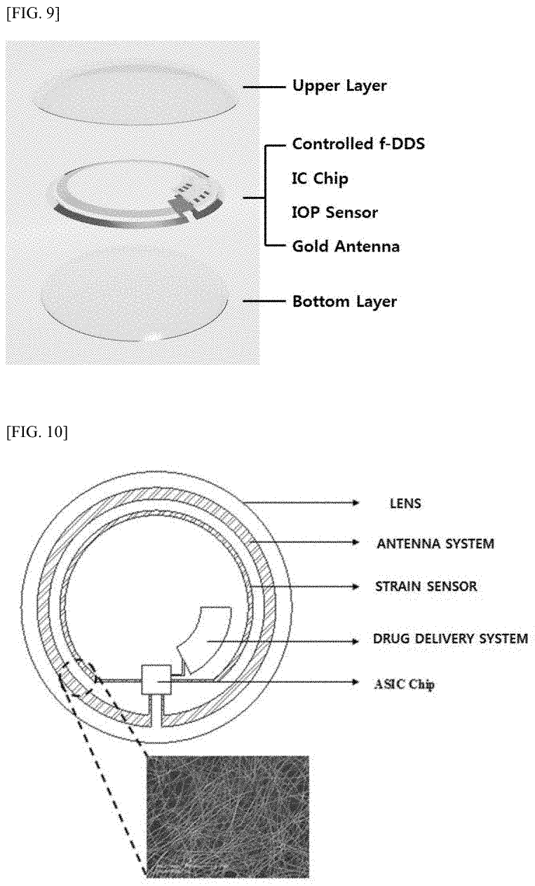

FIGS. 9 and 10 are images of a wirelessly driven contact lens according to the present invention. As shown in FIGS. 9 and 10, it can be confirmed that the wirelessly driven contact lens includes a strain sensor, an antenna, and a drug reservoir (drug delivery system), and the strain sensor, the antenna, and the drug reservoir are formed on a transparent substrate. In addition, it can be seen that the strain sensor and the antenna are made of Ag@Au core-shell NWs.

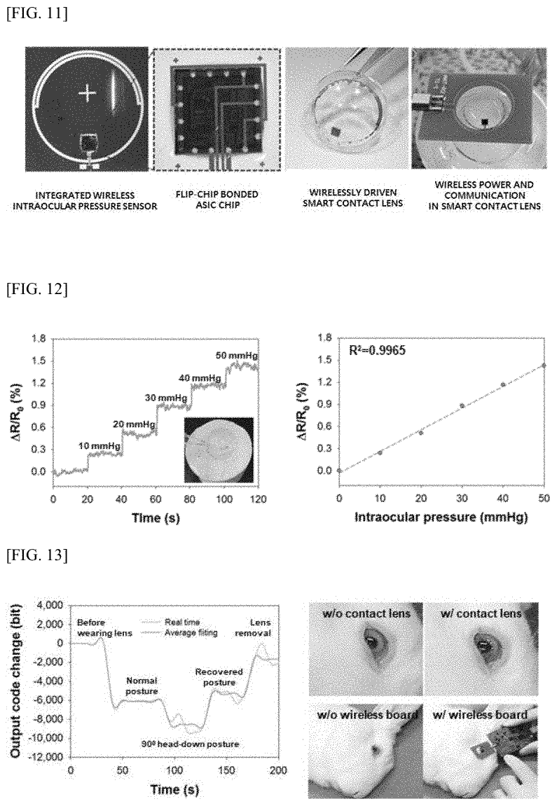

In addition, FIG. 11 shows photographs showing a state in which a strain sensor using prepared Ag@Au core-shell NWs is inserted into a lens.

### Experimental Example 1: Response of Strain Sensor

Changes in resistance and current according to a change in intraocular pressure were measured on the contact lens manufactured in Manufacturing Example 3. The contact lens includes the strain sensor formed in Manufacturing Example 1 described above.

A potentiostat and the strain sensor were connected with a copper wire, and then a voltage of 0.6 V was applied.

In order to allow intraocular pressure to be changed between 0 mmHg and 50 mmHg, two needles were inserted into an eyeball model, one needle was connected to a syringe pump, and the other needle was connected to a manometer.

After the manufactured contact lens was bonded to the eyeball model, changes in resistance and current according to a change in intraocular pressure were measured (see FIG. 12). Specifically, after pressure was maintained for about 20 seconds every 10 mmHg, the pressure was increased to observe the changes in resistance and current according to intraocular pressure.

In addition, data about a change in resistance of the strain sensor according to a posture of a rabbit was wirelessly measured (see FIG. 13).

FIGS. 12 and 13 show results in which a change in resistance according to a change in intraocular pressure was measured using a strain sensor through in vitro and in vivo experiments using a contact lens. As shown in FIGS. 12 and 13, it can be confirmed that the change in intraocular pressure can be easily measured using the strain sensor formed in the contact lens.

### Experimental Example 2: Release of Drug from Drug Reservoir

A drug (timolol or latanoprost) was loaded into the drug reservoir of the contact lens manufactured in Manufacturing Example 3 to measure a degree of release of the drug.

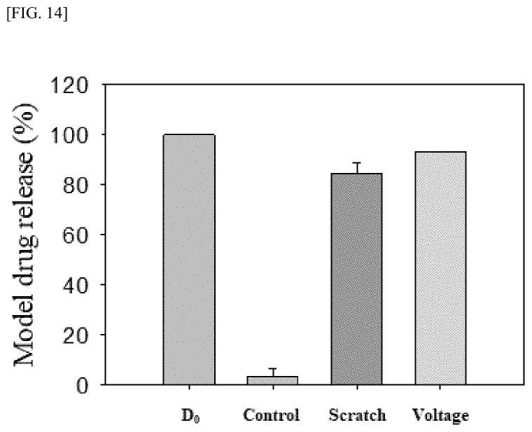

FIG. 14 is a graph showing measured absorbance of a drug released from a drug reservoir.

As shown in FIG. 14, it can be confirmed that the drug is easily released according to an electrical signal from the drug reservoir.

### INDUSTRIAL APPLICABILITY

In the present invention, through the strain sensor and the drug delivery system, intraocular pressure can be measured in real time, and a therapeutic drug can be appropriately released from a contact lens. Accordingly, personal therapy is possible through a feedback treatment system based on an intraocular pressure state of a glaucoma patient.

1. A wirelessly driven contact lens for monitoring intraocular pressure and treating glaucoma in a glaucoma patient, the wirelessly driven contact lens comprising:
a strain sensor which is transparent and measures intraocular pressure of a subject; and
a drug reservoir,
wherein the strain sensor and the drug reservoir are formed on a transparent substrate,
wherein the strain sensor measures a change in resistance due to a change in intraocular pressure, and
wherein when an abnormality is detected in the change in intraocular pressure, a drug is released from the drug reservoir.

2. The wirelessly driven contact lens of claim 1, wherein the contact lens is based on at least one selected from the group consisting of an elastomer such as a silicone elastomer, a silicone hydrogel, and a polymer hydrogel of poly(2-hydroxyethyl methacrylate) (PHEMA), polyvinylpyrrolidone (PVP), poly(lactic acid-glycolic acid) (PLGA), or polyvinyl alcohol (PVA).

3. The wirelessly driven contact lens of claim 1, wherein the transparent substrate includes at least one selected from the group consisting of parylene C, polydimethylsiloxane (PDMS), a silicone elastomer, polyethylene terephthalate (PET), and polyimide (PI).

4. The wirelessly driven contact lens of claim 1, wherein the strain sensor includes:
a nanomaterial layer formed on the transparent substrate; and
a passivation layer formed on the nanomaterial layer.

5. The wirelessly driven contact lens of claim 4, wherein nanomaterials included in the nanomaterial layer include at least one selected from the group consisting of zero-dimensional materials that are nanoparticles, one-dimensional nanomaterials that are nanowires, nanofibers, or nanotubes, and two-dimensional nanomaterials that are graphene, MoS₂, or nanoflakes.

6. The wirelessly driven contact lens of claim 5, wherein the nanomaterials have biocompatibility.

7. The wirelessly driven contact lens of claim 4, wherein the passivation layer includes at least one selected from the group consisting of parylene C, polydimethylsiloxane (PDMS), a silicone elastomer, polyethylene terephthalate (PET), and polyimide (PI).

8. The wirelessly driven contact lens of claim 1, wherein a structure of the strain sensor comprises a circle or a straight line and entirely or partially surrounds the cornea of an eyeball.

9. The wirelessly driven contact lens of claim 1, wherein the drug reservoir includes an electrode pattern which includes gold and is formed on a portion of a surface of the transparent substrate and a drug well layer which is formed on the electrode pattern and includes one or more drug wells with a shape that is recessed so as to face outward,
wherein perforations are formed in the transparent substrate, and
wherein the electrode pattern covers the perforations.

10. The wirelessly driven contact lens of claim 9, wherein the drug included in the drug well includes a drug for treating glaucoma or includes a drug carrier for releasing a drug and a drug-release-controlling material.

11. The wirelessly driven contact lens of claim 1, further comprising a circular antenna configured to transmit and receive power and signals to and from the outside through induced current and electromagnetic resonance,
wherein the circular antenna is formed on the transparent substrate.

12. The wirelessly driven contact lens of claim 11, wherein the antenna includes at least one selected from the group consisting of metal thin film materials, zero-dimensional materials that are nanoparticles, one-dimensional nanomaterials that are nanowires, nanofibers, or nanotubes, and two-dimensional nanomaterials that are graphene, MoS₂, or nanoflakes.

13. A method of manufacturing the wirelessly driven contact lens for monitoring intraocular pressure and treating glaucoma in a glaucoma patient according to claim 1, the method comprising:
forming a sacrificial layer soluble in water on a handling substrate;
forming a transparent substrate on the sacrificial layer;
forming a strain sensor and a drug reservoir on the transparent substrate; and
transferring the transparent substrate, on which the strain sensor and the drug reservoir are formed, into a contact lens.

14. The method of claim, 13, wherein the sacrificial layer includes at least one selected from the group consisting of polyvinyl alcohol (PVA) and dextran.

15. The method of claim 13, wherein the forming of the strain sensor on the transparent substrate includes:
forming a mask material for patterning on the transparent substrate;
patterning a sensor and a circuit by coating nanomaterials on the transparent substrate, on which the mask material is formed, through a lift-off process; and
forming a passivation layer on the sensor and circuit that are patterned.

16. The method of claim 13, wherein the forming of the drug reservoir on the transparent substrate includes:
forming an electrode pattern including gold on a portion of a surface of the transparent substrate; and
forming a drug well layer including one or more drug wells on the electrode pattern.

17. The method of claim 16, wherein one or more perforations are formed in the transparent substrate on which the drug reservoir is formed,
wherein the electrode pattern covers the perforations, and
wherein the perforations are formed before or after the electrode pattern is formed on the transparent substrate.

18. The method of claim 16, wherein the drug well layer including the drug wells includes at least one selected from the group consisting of polydimethylsiloxane (PDMS), a silicone elastomer, polyurethane acrylate (PUA), and an SU8.

19. The method of claim 13, further comprising forming an antenna on the transparent substrate.

20. A wirelessly driven system for monitoring intraocular pressure and treating glaucoma in a glaucoma patient, the system comprising:
a wirelessly driven contact lens including a strain sensor, which is transparent and measures intraocular pressure of a subject, and a drug reservoir; and
smart glasses configured to wirelessly transmit or receive an electrical signal and control driving of the strain sensor and the drug reservoir of the wirelessly driven contact lens,
wherein the strain sensor and the drug reservoir are formed on a transparent substrate,
wherein the strain sensor measures a change in resistance using electrical connection and disconnection between nanomaterials according to a change in curvature of an eyeball due to a change in intraocular pressure, and
wherein when an abnormality is detected in the change in intraocular pressure, a drug is released from the drug reservoir.

21. A method of treating glaucoma based on an intraocular pressure state using the system according to claim 20, the method comprising:
applying, by a strain sensor in a contact lens, a constant voltage to an eyeball of a subject for a predetermined measurement time and wirelessly measuring a change in current according to a change in resistance; and
when the change in current due to a change in intraocular pressure of the eyeball of the subject is measured to be greater than or equal to a set range, dissolving gold of an electrode pattern sealing a drug well of a drug reservoir through chlorine ions to form AuCl⁴⁻ and opening the drug reservoir.

22. The method of claim 21, wherein the strain sensor is driven through an electrical signal transmitted from smart glasses, and
the strain sensor from which the signal is received measures the change in resistance according to the change in intraocular pressure and the change in current according to the change in resistance and transmits results of the changes to the smart glasses through wireless communication.

23. The method of claim 21, wherein the drug reservoir is driven through an electrical signal transmitted from smart glasses,
wherein the smart glasses analyze the change in resistance or the change in current transmitted through the strain sensor, and
wherein when an abnormality is detected in the change in intraocular pressure, the smart glasses transmit the electrical signal to the drug reservoir, and
the drug reservoir from which the signal is received opens.

24. The method of claim 21, wherein power generated from a wireless electric coil of smart glasses is received by a wireless electric antenna of the wirelessly driven contact lens, and
wherein the power that is received is used to drive a sensor and a drug delivery system under control of an integrated circuit (IC) chip.
